(19) United States
(12) Patent Application Publication
(10) Pub. No.: US 2002/0120223 A1
(43) Pub. Date: Aug. 29, 2002

(54) Blood purifier featuring micro-euthanasia of undesirable blood organisms

(76) Inventor: Carl C. Kling, Cos Cob, CT (US)

Correspondence Address: Carl C. Kling, PO Box 305, Hawthorne, NY, 10532-0305, US

(21) Appl. No.: 09/789,059

(22) Filed: Feb. 20, 2001

(51) Int. Cl. A61M 37/00; B01D 33/15

(52) U.S. Cl. 604/5.01; 604/6.08; 210/782

(57) ABSTRACT

A blood purifying system, in which a patient is connected to a blood centrifuge, and certain components of the patient's blood are forwarded and further treated at a purifying station by a microscopic automatic review to identify undesirable organisms, and with a controlled laser blast focused on such identified undesirable organisms to destroy them as they pass through the purifying station. A patient having a suspected condition involving blood clots or undesirable organisms in the patient's bloodstream is connected so that the blood flows slowly through a purifying station where the stream is spread across a significant area in a very smooth layer enabling inspection by a laser scanner and pattern recognition system. When an undesired particle is detected, there is a double check of the pattern recognition, and, assuming recognition as undesirable, the organism is destroyed by a focused laser blast. Wherever the destruction particulates are expected to be appropriately heavier or lighter, the bloodstream portion is diverted to another centrifuge to remove the resulting particles before returning the blood to the patient.

### BACKGROUND OF THE INVENTION

(1) Field of the invention

This invention relates to a blood purifying system, and particularly relates to a blood purifying system in which a patient is connected to a blood centrifuge, and certain components of the patient's blood are forwarded and further treated by a microscopic automatic review to identify undesirable organisms, with a controlled laser blast focused on such identified undesirable organisms to destroy them as they pass through the purifying station.

(2) Description of Related Art

Dialysis is the most common procedure in which a patient is connected to a blood system for a period of time while the blood itself is purified, essentially by differential osmotic pressure across a semi-permeable membrane. Dialysis mimics the purifying technique of the human kidneys, by a sort of mechanical kidney.

Another blood purifying procedure is the multiple-transfusion procedure used in treating the “blue-baby” syndrome in which maternal antibodies have improperly passed through a wound in the placenta and are attacking the bay's blood. The baby's blood is replaced by progressively greater dispersion of new blood into the baby's circulatory system—until the antibodies are so diminished in number that they no longer present a risk to the baby.

Blood banks have become common. They withdraw blood from healthy patients and forward it as the most common human organ transplant, usually simply to replace blood lost through trauma. There is a limit of how much blood a donor can give, however, and there has been a continuing search for the solution to the limit problem.

One solution to the limit problem is the blood separation centrifuge. These are deployed throughout most systems of modern hospitals. These centrifuges have special devices which permit a flow of blood through the high-speed centrifuge while the blood flows through lumen tubes which do not rotate. Such centrifuge systems are used to make separations of blood components and deliver the selected components usually to test tubes for use or analysis. One such use is platelet removal.

Still another situation in which a patient is connected to a blood system is a blood recovery during surgical operations. Such systems recover blood spilling from the surgical wound, disinfect it and return it to the circulatory system of the same patient.

The search for cures of blood-borne diseases continues.

### BRIEF SUMMARY OF THE INVENTION

This invention Is a disease control technique for the identification and selective destruction of undesirable micro-organisms in the human bloodstream. There are certain viruses, particularly retro-viruses, and some microbes, as well as some abnormal human blood and lymph cells, which resist selective control by drugs. These micro-organisms have distinctive configurations and can be identified under high magnification microscopes, using computer recognition techniques. Once identified, even in a moving stream, the undesirable particulates can be killed or, if already dead, broken up by a controlled laser beam.

It is the object of the invention to provide a system for monitoring micro-organism particulates in the human blood stream and destroying unwanted micro-organisms.

A feature of the invention is the use of recognition techniques on flowing human blood to identify unwanted micro-organisms for destruction.

An advantage of the invention is that the person being treated has no discomfort, since the monitoring and purifying of the blood is done outside the body.

Other objects, features and advantages of the invention will be apparent from the following written description, claims, abstract and the annexed drawings.

This invention places the patient at a treatment station, with a blood recirculating transfer-and-return centrifuge blood fractionating subsystem passing the appropriate fractions of the patient's blood through a transparent treatment chamber dimensioned and coated to isolate individual blood particles for identification. Once identified, with a blood fraction still flowing in the treatment chamber, undesired micro-organisms, or other undesirable particles such as blood clots, are identified and euthanized or otherwise destroyed. The treated blood fraction is then re-centrifuged to remove treated particles, or recirculated to the patient.

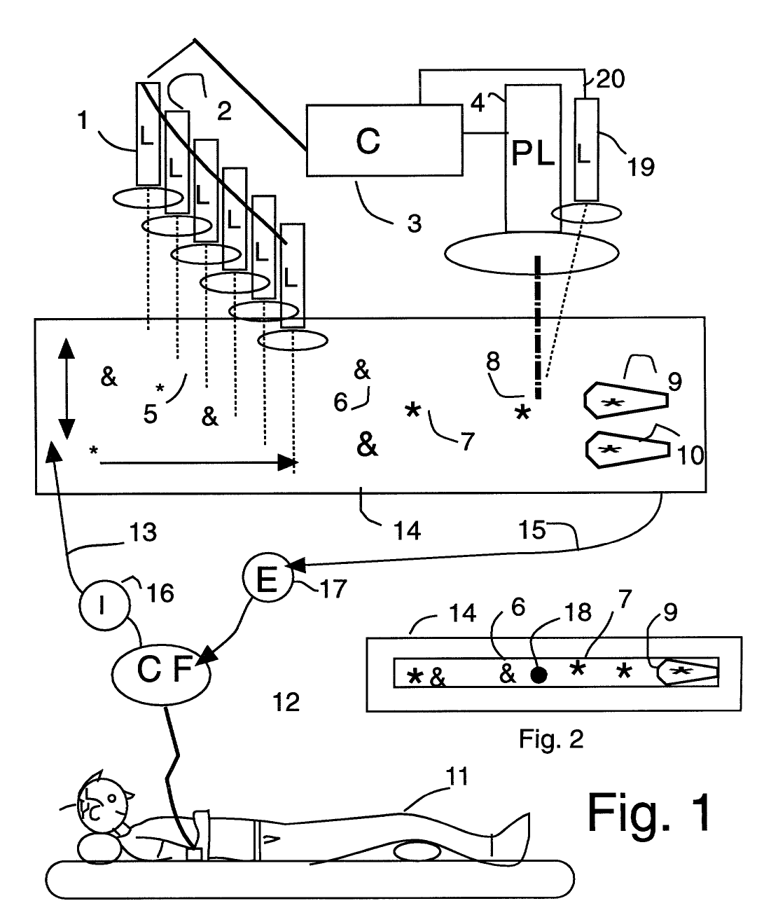

FIGS. 1 & 2 show the invention. A set 1 of laser scanners, including laser scanner 2 for later discussion, views the fractionated blood particles as they pass, and supplies viewing data to computer control unit 3 which compares the individual blood particles to recognition norms. A micro-organism particle recognized as a disease particle has its coordinates determined, is monitored by dynamic automatic monitoring/locating means, and when it comes within range, is subjected to the beam of power-laser 4 which kills the micro-organism or breaks up the particle. A disease micro-organism denoted schematically by an asterisk (*) 5 differs from healthy blood particle (&) 6, of course also shown schematically. Disease * micro-organisms 7 and 8 have been identified as such and are scheduled by computer 3 for micro-euthanasia. Disease micro-organism 8 is shown being euthanized by the beam from power-laser 4. Disease micro-organisms 9 and 10 are shown schematically as already euthanized by the beam from power-laser 4.

Patient 11 is shown connected to centrifuge 12, which may be a Baxter Travenol blood fractionating centrifuge which is geometrically configured to permit centrifuge operation of the patient's blood while the patient is connected. The appropriate blood fraction passes from centrifuge (CF) 12, via lumen tube 13, to and through ultra-thin treatment chamber 14. Treated blood then passes via exit lumen tube 15 back to the centrifuge 12 and back to the patient 11. If considered advantageous, the blood fraction may bbe again centrifuged to remove euthanasia detritus or particle detritus if such detritus is susceptible to centrifuging. As needed, the blood fraction to be treated is diluted at induction unit 16. The dilutant may be drained off at electrophoresis unit 17 or simply retained in the centrifuge as an easily-separated blood fraction, without actually being returned to the patient 11.

FIG. 2 shows treatment chamber 14 in cross-section, not to scale. An X-y stage moves treatment chamber 14 with respect to the set of laser scanners 1. The interior 18 is very thin, to isolate individual blood particles and micro-organisms.

A final re-check laser scanner micro-camera 19 is focused identically with power laser 4 to make sure that the micro-organism selected for euthanasia has not migrated from its expected location, and to review the results after the euthanasia beam. The control unit 3 delays the killing blast from power laser 4 until satisfied from the output of last camera 19 that the micro-organism is in fact a candidate for micro-euthanasia.

Much like the blue-baby full-blood-supply transfusion technique, this technique may effectively treat the entire blood supply of the patient during one prolonged session. In most cases, the detritus from the euthanized micro-organisms 9 and 10 will be harmless, or perhaps even be beneficial as being a sort of killed-virus site for activating T-cell and leukocyte production.

It is preferable that the patient remains comfortable at the treatment station, because of the connection to the blood centrifuge, but it is not necessary that the patient remain absolutely stationary. At the magnifications involved, the micro-organisms are monitored as they march past the reviewing station. The undesirable micro-organisms are identified and located, and their motions are predicted with a set of coordinates which the computer in the control unit keeps updated. It is not ordinarily necessary to have any relative movement between micro-euthanizing power laser 4 and the blood purifying station 14, since the micro-organisms are continuously moving past the station. Mounting a projection lens in the power laser light path, on a rocker or on a slide, permits the change of laser focus to zoom up or to choke down the radiation pattern for better access to the micro-organism to be micro-euthanized or particle to be shattered. Laser focus can also be altered electronically. There can also be a change of beam direction in a scan line across the chamber or beam direction in a scan line parallel to the axis of the chamber: it is also advantageous in some situations to re-centrifuge the targeted liquid component to eliminate the detritus of targeted organisms or particles.

### Implementation

Implementation of the preferred embodiment will require a blood centrifuge, a blood reviewing station with a high-power microscope with input to a computer, a high-speed computer with pattern recognition capability and operator intervention capability, as well as facilities to make the patient comfortable. It may be advisable to mount the blood reviewing station on a precision X-Y stage. Suitable lasers and stages are described in one or more of the following US patent applications:

U.S. Ser. No. 09/531,629, Kling, CONFIGURED-HOLE HIGH-SPEED DRILLING SYSTEM FOR MICRO-VIA PATTERN FORMATION, AND RESULTING STRUCTURE, Feb. 21, 2000;

U.S. APPLICATION OF JAIN Eet al., DUAL-BEAM MATERIAL PROCESSING SYSTEM, filed Jan. 17, 2001;

U.S. Pat. No. 6,040,552, HIGH-SPEED DRILLING SYSTEM FOR MICRO-VIA FORMATION, AND RESULTING STRUCTURE, JAIN et al., issued Mar. 21, 2000

1. A liquid purifying system in which a certain selected liquid component, which may include a number of discrete particles, some of which particles may be identifiable as benign and some of which particles may be identifiable as undesirable or suspect, is forwarded for treatment at a liquid component reviewing station, characterized by
means to divert a selected portion of said selected liquid component to an ultra-shallow inspection and treatment station;
means to scan parameters of said selected liquid component in said liquid component treatment station in a microscopic automatic review to identify, locate and track an individual particle 1 . . . n;
means to determine whether such individual particle 1 . . . n is benign, suspect or undesirable; and
means to focus a controlled laser blast on such individual organism (1 . . . n) if determined to be an undesirable particle, to change the characteristics of said undesirable particle as it passes through said reviewing station.

2. A liquid purifying system according to claim 1, further characterized in that
said means to determine whether such individual organism 1 . . . n is benign, suspect or undesirable is adapted to store parameters of said organism for future consideration.

3. A liquid purifying system according to claim 2, further characterized in that
said means to identify, locate and track an identified undesirable organism comprises means to identify a suspect organism and present parameters of such suspect organism for further identification as undesirable organism 1 . . . n; and
said means to present parameters includes means to identify such suspect organism to said means to identify, locate and track a suspect organism includes means to recall the parameters of such suspect organism for further identification automatically.

4. A liquid purifying system according to claim 3, further characterized in that
said particle is a living organism.

5. A liquid purifying system according to claim 3, further characterized in that
said liquid is a fraction of human blood.

6. A liquid purifying system according to claim 4, further characterized in that
said particle is a blood clot.

7. A blood purifying system in which a patient is connected to a blood centrifuge, and certain selected component liquids of the patient's blood, including a number of discrete organisms, some of which organisms may be identifiable as benign and some of which organisms may be identifiable as undesirable or suspect, are forwarded for treatment at a liquid component reviewing station, characterized by
means to divert a selected component portion of the patient's bloodstream to an ultra-shallow inspection and treatment portion of said blood component reviewing station;
means to scan parameters of said selected component in said liquid component treatment station in a microscopic automatic review to identify, locate and track an individual organism 1 . . . n;
means to determine whether such individual organism 1 . . . n is benign, suspect or undesirable; and
means to focus a controlled laser blast on such individual organism (1 . . . n) if determined to be an undesirable organism, to euthanize said undesirable organism as said blood component passes through said treatment station back to the patient.

9. A blood purifying system according to claim 8, further characterized in that
a second centrifuge is included to remove the detritus of said undesirable organisms as said blood component passes out of said reviewing station back to the patient.

10. A blood purifying system according to claim 8, further characterized in that
a dilutant is added to said blood component prior to said treatment station and a second centrifuge is included to remove the detritus of said undesirable organisms and said dilutant as said blood component passes out of said reviewing station back to the patient.

11. A blood purifying system according to claim 8, further characterized in that
a dilutant is added to said blood component prior to said treatment station and a second centrifuge is included to said dilutant as said blood component passes out of said reviewing station back to the patient.

12. A liquid purity testing system in which a certain selected liquid component, which may include a number of discrete particles, some of which particles may be identifiable as benign and some of which particles may be identifiable as undesirable or suspect, is forwarded to a liquid component reviewing station, characterized by
means to divert a selected portion of said selected liquid component to a transparent window in an ultra-shallow portion of an inspection station;
means to scan parameters of said selected liquid component in said liquid component inspection station in a microscopic automatic review to identify an individual particle 1 . . . n;
means to determine whether such individual particle 1 . . . n is benign, suspect or undesirable; and
means to provide test results.

13. A liquid purity testing system according to claim 12, further characterized in that
said means to determine whether such individual particle 1 . . . n is benign, suspect or undesirable and to provide at least one count related to number of undesirable particles.
